(19) United States
(12) Patent Application Publication
(10) Pub. No.: US 2005/0208542 A1
(43) Pub. Date: Sep. 22, 2005

(54) Methods of genotoxicity screening, and biosensors

(76) Inventors: James F. Rusling, Storrs, CT (US); John B. Schenkman, Simsbury, CT (US); Liping Zhou, Cambridge, MA (US); Jing Yang, Urbana, IL (US)

Correspondence Address: CANTOR COLBURN, LLP, 55 GRIFFIN ROAD SOUTH, BLOOMFIELD, CT, 06002, US

(21) Appl. No.: 11/031,531

(22) Filed: Jan. 7, 2005

Related U.S. Application Data
(60) Provisional application No. 60/535,673, filed on Jan. 8, 2004.

(51) Int. Cl. C12Q 1/68; C12M 1/34

(52) U.S. Cl. 435/6; 435/287.2

(57) ABSTRACT

A biosensor is described comprising an electrode, a protein layer (s), and a double-stranded polynucleotide layer (s). Also included do arrays, kits, and instruments comprise the biosensors. The biosensors may be used to detect double-stranded polynucleotide damage resulting from metabolism of a test compound, such as a drug candidate, by the protein in the protein layer.

### CROSS REFERENCE TO RELATED APPLICATIONS

This application claims priority from Provisional Application Ser No. 60/535,673, filed Jan. 8, 2004, which is incorporated herein by reference in its entirety.

### STATEMENT REGARDING FEDERALLY SPONSORED RESEARCH & DEVELOPMENT

The U.S. Government has certain rights in this invention pursuant to Grant No. ES03154 awarded by the National Institute of Environmental Health Sciences.

### BACKGROUND

Medical abnormalities resulting from exposure to toxic chemicals constitute a critical public health problem in the modern world. One mechanism of chemical toxicity involves bioactivation of toxic chemicals by enzyme-mediated oxidation in the liver. Lipophilic molecules bioactivated in this manner often damage DNA. For example, damage of DNA by cytochrome P450-derived metabolites of lipophilic pollutants and drugs in the mammalian liver may lead to structural changes in the DNA. Methods for detecting such DNA damage could serve as effective in vitro screens for the toxicity of new organic chemicals at an early point in their commercial development.

Conventional toxicity screening of new chemicals may proceed through microbiological testing and animal testing, and is both expensive and time consuming. It is thus desirable to develop new methods of screening chemicals for toxicity, particularly methods that are less costly and time consuming than conventional microbiological methods, for example.

Electrochemical methods have the potential to provide a rapid, relatively inexpensive approach to detecting DNA damage and hybridization and may provide an alternative to conventional toxicity screening. Adenine and guanine bases in DNA may undergo electrochemical oxidations which give much larger electrochemical signals when the DNA is single stranded or chemically damaged. In the double helix form, nucleic acid bases are protected, and minimal oxidation occurs. It has been demonstrated that ultrathin films of double stranded (ds)-DNA and polycations on electrodes may be used for detection of DNA damage from a known damage agent, styrene oxide, a liver metabolite of styrene. Styrene oxide forms up to eleven covalent adducts with guanine and adenine moieties that disrupt the double helix. Damaged DNA in the films, upon incubation with styrene oxide, was detected by square wave voltammetry using catalytic oxidation with an oxidizing agent. In addition, it has been demonstrated that liver metabolites such as those of styrene may be accurately and efficiently produced by thin films containing liver enzymes such as the cytochrome P450s.

### SUMMARY

A biosensor comprises an electrode; a first polynucleotide layer comprising a double-stranded polynucleotide, wherein the electrode and the polynucleotide layer are in electrochemical communication; and a first protein layer in chemical communication with the polynucleotide layer, wherein the protein layer comprises a protein that converts a genotoxic compound into a metabolite that damages double-stranded polynucleotide.

A method of detecting a genotoxic compound comprises contacting the above-described biosensor with a test compound; and detecting polynucleotide damage in the polynucleotide layer resulting from said contacting. The presence of polynucleotide damage in the biosensor is indicative of the toxicity of the test compound.

### BRIEF DESCRIPTION OF THE DRAWINGS

FIGS. 1-4 show embodiments of biosensors.

FIG. 5 shows a schematic of a biosensor interacting with styrene oxide as a model test compound.

FIG. 6 shows quartz crystal microbalance (QCM) frequency shifts for cycles of alternate myoglobin (Mb)/double-stranded-DNA (ds-DNA) and cytochrome (cyt) P450_{cam}/salmon testes (ST) ds-DNA adsorption on gold resonators coated with mixed monolayers of mercaptoproionic acid/mercaptopropanol as first layer and poly(diallyldimethylammonium chloride) (PDDA) as second layer.

FIG. 7 shows square wave voltammetry of PDDA/ds-DNA(/Mb/ds-DNA)₂ films on rough pyrolytic graphite (PG) in pH 5.5 buffer containing 50 μM Ru(bpy)₃²⁺ before and after incubations at 37° C. with 2% styrene (no styrene in controls) and 0.2 mM H₂O₂ in aerobic buffer solution. (SWV Amplitude: 25 mV; Frequency: 15 Hz; Step: 4 mV).

FIG. 8 illustrates the influence of reaction time with 2% styrene+0.2 mM H₂O₂ on catalytic peak current in 50 μM Ru(bpy)₃²⁺ for PDDA/ds-DNA(/Mb/ds-DNA)₂ films using calf thymus (CT)-DNA (◯) and ST-DNA (●) (5-15 trials per data point).

FIG. 9 illustrates the influence of reaction time with 2% styrene+0.2 mM H₂O₂ on the average catalytic peak current for PDDA/ds-ST-DNA(/cyt P450_{cam}/ds-ST-DNA)₂ films (●) in 50 μM Ru(bpy)₃²⁺ solution; control (◯).

FIG. 10 shows the SWV of PDDA/ST-ds-DNA(Mb/ST-ds-DNA)₂ films on rough pyrolytic graphite (PG) reacted at 37° C. with 4% styrene and 2 mM H₂O₂ at pH 5.5, then transferred to 20 μM Co(bpy)₃³⁺ in pH 5.5 buffer.

FIG. 11 illustrates the influence of reaction time with 4% styrene+2 mM H₂O₂ for PDDA/ds-DNA(/Mb/ds-DNA)₂ films using CT-DNA (●) and ST-DNA (◯) and for PDDA/ds-CT-DNA(cyt P450_{cam}/ds-CT-DNA)₂ (Δ) on peak current in 20 μM Co(bpy)₃³⁺.

FIG. 12 shows partial capillary electropherograms showing the possible peaks of DNA-styrene oxide adducts of enzyme hydrolyzed (PDDA/ds-ST-DNA)₂ films after incubation with styrene oxide (S.O.) for 48 hr at 37° C., in comparison to samples of guanosine (dG) and adenosine (dA) after reaction with styrene oxide.

FIG. 13 shows HPLC with UV detection showing possible peaks of DNA-styrene oxide adducts from hydrolyzed DNA after incubation of intact ds-ST-DNA with styrene oxide for 48 hours at 37° C.

FIG. 14 shows HPLC-MS for hydrolyzed DNA after incubation of intact ds-ST-DNA with styrene oxide for 48 hours at 37° C. The top curve for M/e 388 corresponds to deoxyguanosine (dG)-styrene oxide adducts, and the middle curve for M/e 372 corresponds to deoxyadenosine (dA)-styrene oxide adducts.

FIG. 15 shows multiple reaction monitoring (MRM) of hydrolyzed DNA after incubation of intact ds-ST-DNA with styrene oxide for 48 hours at 37° C. The top curve corresponds to detection of dG-styrene oxide adducts and the bottom curve corresponds to detection of dA-styrene oxide adducts.

FIG. 16 shows the difference square wave voltammograms of Ru/Mb/DNA films on rough PG electrodes in the (b) absence and (c) presence of saturated oxygen and (a) for a CIRu-PVP electrode with no DNA or Mb.

FIG. 17 shows the influence of incubation time on catalytic current ratio of Ru/Mb/DNA films treated at pH 5.5 with 2 mM H₂O₂ and saturated styrene (●), 2 mM H₂O₂ and saturated toluene (▾), and 0.2 mM H₂O₂ and saturated styrene (◯).

FIG. 18 shows SWV and ECL response for Ru-PVP alone (◯), (Ru-PVP/PSS)₂ (▴), (Ru-PVP/poly[A])₂ (⋄), and (Ru-PVP/poly[G])₂ (▪).

FIG. 19 shows SWV and ECL response for (a) Ru-PVP, (b) (Ru-PVP/poly-[G]/poly[C])₂ and (c) (Ru-PVP/poly[G])₂ films.

FIG. 20 shows the SWV and ECL for (a) (Ru-PVP-ds-CT DNA)₂ films and (b) (Ru-PVP/ss-CT DNA)₂ films and (c) SWV only for Ru-PVP film with no DNA.

FIG. 21 shows SWV and ECL responses for (Ru-PVP/ds-CT DNA)₂ films after incubation at 37° C. with saturated styrene oxide.

FIG. 22 shows the influence of incubation time with styrene oxide (●), toluene (▴), and buffer alone on average ECL signal for (Ru/ds-CT DNA)₂ films.

FIG. 23 shows the influence of incubation time with styrene oxide (●), toluene (▴), and buffer alone on average SWV signal for (Ru/ds-CT DNA)₂ films.

FIG. 24 shows SWV and ECL response for (Ru-PVP/poly[G])₂ films: (a) no incubation, (b) incubated with saturated toluene control for 10 minutes, and (c) incubated with saturated styrene oxide for 10 minutes.

### DETAILED DESCRIPTION

Disclosed herein are biosensors and methods for toxicity screening, i.e., screening for the ability of the test compound and/or its metabolites to damage double-stranded polynucleotides. While the biosensors and methods will be described for double-stranded DNA, it should be understood that other double-stranded polynucleotides such as double-stranded RNA may be employed. This type of screening is referred to herein as genotoxicity screening. Genotoxins may, for example, cause DNA mutations, neoplasms, and/or tumors. The biosensor comprises an electrode, one or more protein layers, and one or more DNA layers comprising double-stranded DNA. In the biosensor, the DNA layer and the electrode are in electrochemical communication, and the protein layer and the DNA layer are in chemical communication. A method of toxicity screening comprises contacting a test compound with the biosensor and detecting damage in the double-stranded DNA. In the method, the protein layer contains a protein, for example an enzyme, capable of bioactivating a genotoxic test compound, e.g., converting the test compound to its metabolites, or acting on the test compound to produce one more genotoxic metabolites. If the test compound is capable being bioactivated by the protein layer, the metabolites thus formed then interact with the double-stranded DNA in the DNA layer and may cause damage, e.g., chemical adducts and/or breaks, in the double-stranded DNA in the biosensor. The double-stranded DNA damage mimics DNA damage that may occur in the liver. The DNA damage may then be detected by electrochemical methods such as, for example, voltammetry and/or electrochemiluminescence. A positive signal within a prescribed time period (e.g., about 10 to about 15 minutes) with respect to an unreactive control is indicative of the toxicity of the test compound.

The biosensor comprises an electrode. The term “electrode” refers to an electrical conductor that conducts a current in and out of an electrically conducting medium. The electrode may be present in the form of an array, consisting of a number of separately addressable electrodes, or a dipping electrode such as, for example, an ultramicroelectrode. The electrode comprises an electrically conductive material such as, for example, gold, carbon, tin, silver, platinum, palladium, and combinations comprising one or more of the foregoing materials. Specific electrodes include gold electrodes, carbon electrodes, and tin oxide because of their excellent electrical conductivity and chemical stability. It is to be understood that as used herein, a “layer” may have a variety of configurations, for example rectangular, circular, a line, an irregular dot, or other configuration. A suitable electrode is a pyrolytic graphite disk such as that available as PG from Advanced Ceramics.

The protein layer(s) on the biosensor comprises a protein capable of bioactivating a toxic test compound, e.g., acting on the test compound to produce one or more toxic metabolites. In one embodiment, the protein is an enzyme that converts compounds into metabolites in the liver. In another embodiment, the protein is an enzyme capable of bioactivating a genotoxic test compound, e.g., converting the test compound to its metabolites that damage double-stranded DNA. Liver hepatocytes, for example, express a family of enzymes called cytochromes. One subfamily of cytochromes is known as cytochrome P450. The cytochrome P450 enzyme (CYT 450) family comprises oxidase enzymes involved in the xenobiotic metabolism of hydrophobic drugs, carcinogens, and other potentially toxic compounds and metabolites circulating in blood. Metabolism of a test compound by a CYT P450 enzyme may lead to production of a metabolite capable of inducing DNA damage.

Like cytochrome P450, myoglobin is an iron heme protein. Myoglobin can be used as a less expensive, more readily available alternative to cytochrome P450. Myoglobin, when activated by hydrogen peroxide, catalyzes oxidation via a ferrylmyoglobin radical. Activated myoglobin can, for example, convert styrene to styrene oxide, an agent that can damage DNA.

Other suitable proteins for use in the protein layer include hemoglobin and peroxidases such as, for example, horseradish peroxidase, cytochrome c peroxidase, and glutathione peroxidase. Peroxidases are enzymes which typically contain heme and catalyze the breakdown of peroxides. Combinations of proteins may also be used in a protein layer.

The protein layer(s) may have a thickness of about 2 nanometers to about 100 nanometers. Specifically, the protein layer has a thickness of about 2 nanometers to about 10 nanometers, more specifically about 2 nanometers to about 5 nanometers. The protein layer(s) may have a protein concentration of about 2 micrograms/centimeter² to about 500 micrograms/centimeter². Specifically, the protein layer has a protein concentration of about 3 micrograms/centimeter² to about 100 micrograms/centimeter², more specifically about 4 micrograms/centimeter² to about 20 micrograms/centimeter².

The double-stranded polynucleotide (e.g., DNA) layer(s) of the biosensor comprise a double stranded polynucleotide, such as double-stranded DNA (ds-DNA). The term “nucleoside” refers to a nitrogenous heterocyclic base linked to a pentose sugar, either a ribose, deoxyribose, or derivatives or analogs thereof. The term “nucleotide” relates to a phosphoric acid ester of a nucleoside comprising a nitrogenous heterocyclic base, a pentose sugar, and one or more phosphate or other backbone forming groups; it is the monomeric unit of an oligonucleotide. Nucleotide units may include the common bases such as guanine (G), adenine (A), cytosine (C), thymine (T), or derivatives thereof. The pentose sugar may be deoxyribose, ribose, or groups that substitute therefore.

The terms “polynucleotide” and “nucleotide sequence” refer to a plurality of covalently joined nucleotide units formed in a specific sequence from naturally occurring heterocyclic bases and pentofuranosyl equivalent groups joined through phosphorodiester or other backbone forming groups.

The double stranded polynucleotide may comprise natural or synthetic sequences encoding up to the entire genome of an organism. These polynucleotides can be obtained from, for example, plasmids, cloned DNA or RNA, natural DNA or RNA from a source, such as, for example, bacteria, yeast, viruses, organelles, and higher organisms such as plants and animals. The polynucleotides may be extracted from tissue material or cells, including blood cells, amniocytes, bone marrow cells, cells obtained from a biopsy specimen and the like, by a variety of techniques as described for example by Maniatis et al., Molecular Cloning: A Laboratory Manual, Cold Spring Harbor Laboratory Cold Spring Harbor, N.Y. (1982).

Alternatively, the double-stranded polynucleotide may also be prepared by synthetic procedures. Briefly, oligonucleotides and oligonucleotide analogs may be synthesized, through solid state synthesis of known methodology. The monomeric units may be added to a growing oligonucleotide chain which is covalently immobilized to a solid support. Typically, the first nucleotide is attached to the support through a cleavable linkage prior to the initiation of synthesis. Step-wise extension of the oligonucleotide chain is normally carried out in the 3′ to 5′ direction. When the synthesis is complete, the polymer is cleaved from the support by hydrolyzing the linkage mentioned above and the nucleotide originally attached to the support becomes the 3′ terminus of the resulting oligomer. Nucleic acid synthesizers such as the Applied Biosystems Incorporated 380B are commercially available and their use is generally understood by persons of ordinary skill in the art as being effective in generating nearly any oligonucleotide or oligonucleotide analog of reasonable length which may be desired. Triester, phosphoramidite, or hydrogen phosphonate coupling chemistries are used with these synthesizers to provide the desired oligonucleotides or oligonucleotide analogs.

The double-stranded DNA layer(s) may have a thickness of about 2 nanometers to about 10 nanometers. Specifically, the double-stranded DNA layer has a thickness of about 2 nanometers to about 5 nanometers, more specifically about 2 nanometers to about 3 nanometers. The double-stranded DNA layer(s) may have a DNA concentration of about 3 micrograms/centimeter² to about 100 micrograms/centimeter². Specifically, the double-stranded DNA layer has a DNA concentration of about 3 micrograms/centimeter² to about 50 micrograms/centimeter², more specifically about 3 micrograms/centimeter² to about 10 micrograms/centimeter².

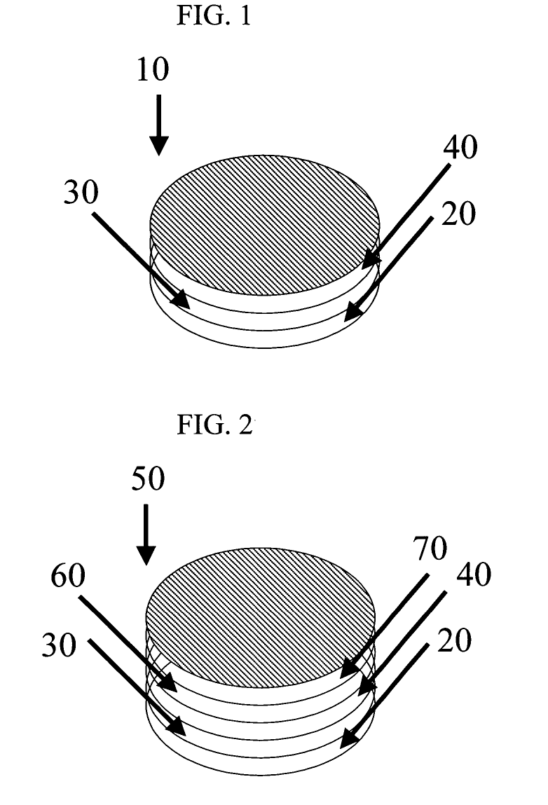

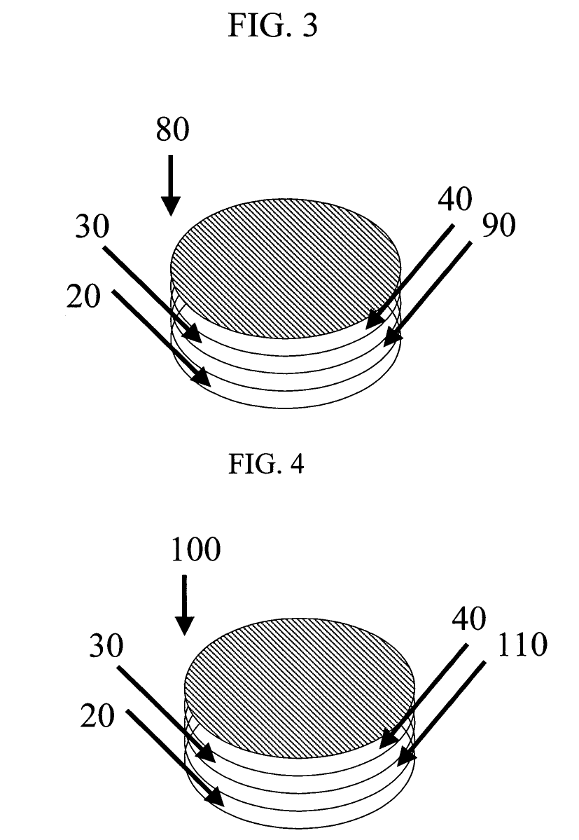

The biosensor may comprise a single protein layer and a single double-stranded DNA layer, or a plurality of alternating protein layers and double-stranded DNA layers. In one embodiment of a biosensor (10), a first side of a first double-stranded DNA layer (30) is disposed on electrode (20). (FIG. 1) A first side of a first protein layer (40) is disposed on, and optionally in intimate contact with, a second side of the first double-stranded DNA layer (30). In another embodiment of a biosensor (50), additional protein and doubled-stranded DNA layers are added to the biosensor. (FIG. 2) In this embodiment of a biosensor, a first side of a second double-stranded DNA layer (60) is disposed on, and optionally in intimate contact with, the second side of the first protein layer (40). A first side of a second protein layer (70) is disposed on, and optionally in intimate contact with, the second side of the second double-stranded DNA layer (60). Other configurations are also possible, for example, the protein layer may be disposed on the double-stranded DNA layer. While, for simplicity, the layers of the biosensor are depicted as individual layers, it should be understood that that layers may not be distinctly separated, e.g., intermingled.

The biosensor may optionally comprise a polyion layer, i.e., a polycation layer or a polyanion layer, wherein the polyion is not a protein or a polynucleotide. Suitable polycations for the polycation layer are poly(diallyldimethylammonium chloride (PDDA), poly(ethylene)imine, or combinations comprising one or more of the foregoing polyions.

The optional polyion layer(s) may have a thickness of about 0.5 nanometers to about 50 nanometers. Specifically, the polyion layer has a thickness of about 1 nanometer to about 5 nanometers, more specifically about 1 nanometer to about 3 nanometers. The polyion layer(s) may have a polyion concentration of about 2 micrograms/centimeter² to about 30 micrograms/centimeter². Specifically, the polyion layer has a polyion concentration of about 2 micrograms/centimeter to about 10 micrograms/centimeter², more specifically about 2 micrograms/centimeter² to about 6 micrograms/centimeter².

When a polyion layer is present, in one embodiment of a biosensor (80), a first side of a polyion layer (90) is disposed on, and optionally in intimate contact with, the electrode (20), and a first side of a first double-stranded DNA layer (30) layer is disposed on, and optionally in intimate contact with, a second side of the polyion layer (90). (FIG. 3) A first side of a first protein layer (40) is disposed on, and optionally in intimate contact with, the second side of the double-stranded DNA layer (30). Additional double-stranded DNA and protein layers can be added to the biosensor. (see FIG. 2, for example).

The biosensor may also comprise an optional redox polymer layer which may be in chemical communication with the double-stranded DNA layer. A redox polymer is a polymer complexed with a redox species such as a transition metal complex. The polymers used to form a redox polymer layer may have nitrogen-containing heterocycles, such as pyridine, imidazole, or derivatives thereof for binding as ligands to the redox species. Suitable polymers for complexation with redox species, such as the metal complexes described below, include, for example, polymers and copolymers of poly(1-vinyl imidazole) (referred to as “PVI”) and poly(4-vinyl pyridine) (referred to as “PVP”), as well as polymers and copolymers of poly(acrylic acid) or polyacrylamide that have been modified by the addition of pendant nitrogen-containing heterocycles, such as pyridine and imidazole. Modification of poly(acrylic acid) may be performed by reaction of at least a portion of the carboxylic acid functionalities with an aminoalkylpyridine or aminoalkylimidazole, such as 4-ethylaminopyridine, to form amides. Suitable copolymer substituents of PVI, PVP, and poly(acrylic acid) include acrylonitrile, acrylamide, acrylhydrazide, and substituted or quaternized 1-vinyl imidazole. The copolymers can be random or block copolymers. Transition metal complexes of redox polymers may be covalently or coordinatively bound with the nitrogen-containing heterocycles (e.g., imidazole and/or pyridine rings) of the polymer. Specific redox polymers include [Ru(bpy)₂(PVP)₁₀](ClO₄)₂, [Ru(bpy)₂poly(4-vinylpyridine)₁₀Cl]⁺, and combinations comprising one or more of the foregoing redox polymers. As used herein, bpy is bipyridyl.

Another type of redox polymer contains an ionically-bound redox species, by forming multiple ion-bridges. This type of redox polymer may include a charged polymer coupled to an oppositely charged redox species. Examples of this type of redox polymers include a negatively charged polymer such as Nafion® (DuPont) coupled to multiple positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. An ionically-bound redox species is a multiply charged, often polyanionic, redox species bound within an oppositely charged polymer.

Another suitable redox polymer includes a redox species coordinatively bound to a polymer. For example, the mediator may be formed by coordination of an osmium, ruthenium, or cobalt 2,2′-bipyridyl complex to poly(1-vinyl imidazole) or poly(4-vinyl pyridine) or by co-polymerization of, for example, a 4-vinyl-2,2′-bipyridyl osmium, ruthenium, or cobalt complex with 1-vinyl imidazole or 4-vinyl pyridine.

The ratio of metal complexes to imidazole and/or pyridine groups of the redox polymer may be about 1:20 to about 1:1, specifically about 1:15 to about 1:2, and more specifically 1:10 to 1:4. Generally, the redox potentials depend, at least in part, on the polymer with the order of redox potentials being poly(acrylic acid)<PVI<PVP.

The optional redox polymer layer(s) may have a thickness of about 1 nanometer to about 200 nanometers. Specifically, the redox polymer layer has a thickness of about 1 nanometer to about 50 nanometers, more specifically about 1 nanometer to about 5 nanometers. The redox polymer layer(s) may have a redox polymer concentration of about 0.1 micrograms/centimeter² to about 100 micrograms/centimeter². Specifically, the redox polymer layer has a redox polymer concentration of about 0.5 micrograms/centimeter² to about 50 micrograms/centimeter², more specifically about 0.5 micrograms/centimeter² to about 10 micrograms/centimeter².

To improve adhesion of the redox polymer layer, the electrode layer may first be coated with poly(sodium 4-styrene-sulfonate) (PSS) prior to coating with the redox polymer to form a PSS/redox polymer layer.

When a redox polymer layer is present, in one embodiment of a biosensor (100), a first side of the redox polymer layer (110) is disposed on, and optionally in intimate contact with, the electrode (20), and a first side of a first double-stranded DNA layer (30) is disposed on, and optionally in intimate contact with, a second side of the redox polymer layer (110). (FIG. 4) A first side of a first protein layer (40) is disposed on, and optionally in intimate contact with, the second side of the first double-stranded DNA layer (30). Additional double-stranded DNA and protein layers can be added to the biosensor.

The protein layer(s), double-stranded DNA layer(s), redox polymer layer(s), polyion layer(s), or a combination comprising one or more of the foregoing layers may be deposited on the electrode layer by a suitable technique for deposition of such layers. One suitable technique is layer-by-layer deposition. The layer-by-layer method permits the fabrication of multilayer film assemblies on solid supports by the spontaneous sequential adsorption of oppositely charged species from aqueous solutions onto solid supports. The driving force for the multilayer film build-up is primarily the electrostatic attraction and complex formation between the charged species deposited.

The protein layer(s), double-stranded DNA layer(s), redox polymer layer(s), and polyion layer(s) may be individually deposited by contacting the electrode layer with a dilute solution comprising the protein, the double-stranded DNA, the redox polymer, or the polyion, respectively. The dilute solution may further optionally comprise buffers, salts, and the like. The electrode may be contacted with the solution for a time sufficient to allow for deposition of the desired amount of the protein, the double-stranded DNA, the redox polymer, or the polyion. Washing with a solvent such as water may be performed between deposition of each successive layer.

In layer-by-layer deposition, the adhesion of the first double-stranded DNA layer or protein layer to the electrode layer may be improved by first depositing a polycation layer on the electrode. Alternatively, or in addition to deposition of a polycation layer, the double-stranded DNA may be derivatized at its 5′-end with a functionalized linker. These linkers include, but are not limited to, thiol- or amine-terminated chains. This process is generally understood by persons of ordinary skill in the art as and is relatively simple, reproducible and can easily be automated.

The deposition of layers may be monitored, for example, with a quartz crystal microbalance (QCM). The frequency change may be measured after the deposition of each layer, for example. A substantially linear increase in the negative of QCM frequency with each additional layer indicates that the film growth is substantially regular with reproducible layers of protein and DNA being added.

While the protein, double-stranded DNA, optional redox polymer and optional polyion layers may be applied as individual layers, they may not exist as discrete, fully distinguishable layers in the finished biosensor. In other words, a slice through the layer in the biosensor may, in fact, show some intermingling between the layers. For example, a protein layer disposed on a double-stranded DNA layer may not appear in the finished sensor as a completely discrete layer, and some intermingling may occur between the protein and the double-stranded DNA. Thus, when it is stated that one layer is disposed on another, this is not meant to indicate that there is either an abrupt or distinct separation between the layers.

The biosensors comprising a protein layer and a double-stranded DNA layer may be used to probe the toxicity of a test compound. A “test compound” as defined herein refers to a chemical, nucleic acid, polypeptide, amino acid, or other compound which is to be tested. Examples of test compounds include, but are not limited to, drug candidates, such as those derived from arrays of small molecules generated through general combinatorial chemistry, as well as any other substances thought to have potential biological activity. A test compound may be labeled such that it and/or its metabolites are easily detected in subsequent analysis. For example, test compounds may be synthesized using radioactive isotopes or fluorescent tags.

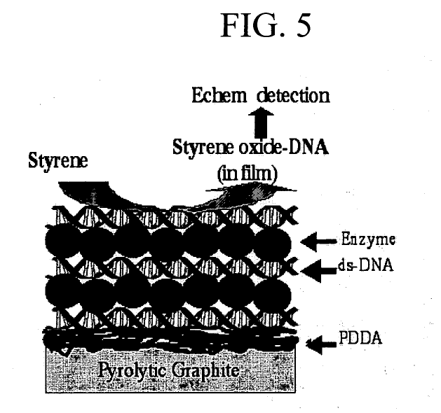

As a model of the interaction of a test compound with the disclosed biosensors, styrene can be employed as a model test compound. Styrene is converted to styrene oxide in the presence of hydrogen peroxide and myoglobin or cytochrome P450. The styrene oxide reacts with double-stranded DNA in the biosensor and produces covalent adducts in the double-stranded DNA, the presence of which can be detected by electrochemical methods. This model reaction is illustrated in FIG. 5.

A “test compound delivery device” refers to devices, apparatuses, mechanisms or tools that are capable of delivering test compounds to an electrode or an array of electrodes. Examples of test compound delivery devices include but are not limited to pipettes or robotic devices (i.e., spotters) well known in the art such as Tecan, PlateMate, or Robbins. A test compound delivery device may be a microfluidic device that delivers a solublized test compound to a test chamber, and specifically to contact an electrode.

The toxicity of the test compound is related to the amount of double-stranded DNA damage caused by the test compound. The term “damage” refers to a departure from the “normal” or ideal structure of a polynucleotide duplex. In the “ideal” structure, all bases are paired with complementary bases, and no nicks, breaks, or gaps occur in the backbones. “Damage” describes the condition in which the conformation of the duplex is perturbed, for example by a nick or break in the backbone, T-T dimerization, DNA adduct formation, and the like. DNA adducts are covalent adducts formed between chemical toxins and DNA. Such adducts may lead to nucleotide substitutions, deletions, and chromosomal rearrangements if not repaired prior to DNA replication.

In practice, a biosensor comprising an electrode layer, a protein layer, and double-stranded DNA layer is contacted with the test compound for a period of time sufficient to determine if the test compound damages the double-stranded DNA. The protein in the protein layer may optionally be activated, for example, by contacting the protein with hydrogen peroxide, organic peroxides (e.g., t-butyl peroxide) or by electrochemical reduction at −0.3 to −0.8 V vs SCE in the presence of oxygen, a process that produces hydrogen peroxide.

After contacting the biosensor with the test compound, a means for detecting double-stranded DNA damage is employed to quantify the amount of double-stranded DNA damage caused by incubation of the biosensor with the test compound. Suitable detecting means include, for example, electrochemical detection by voltammetry and electrochemiluminescence. Voltammetry involves the application of a potential that varies with time and the measurement of the corresponding current that flows between the working and reference electrodes. In square wave voltammetry (SWV), a large amplitude symmetrical square-wave perturbation is used with each cycle of the square wave coinciding with one cycle of an underlying staircase. Current is sampled on both the first and second half of the cycle and can be displayed as a forward current, a reverse current, or the difference between the two measured currents. In the case of double-stranded DNA exposed to a genotoxic metabolite, for example, oxidation peaks may develop as the double-stranded DNA reacts with the metabolite and products such as covalent adducts with guanine and adenine are formed. One possible limitation of direct detection of the SWV peaks is that the signals may be small.

In order to increase the intensity of the SWV peaks, an oxidation probe may be employed in the square wave voltammetry. In one method, catalytic oxidation using transition metal complexes may be employed. The biosensor may be contacted with a solution containing the metal complex prior to detecting, or a redox polymer layer may be included in the sensor. One suitable transition metal complex is ruthenium tris(2,2′-bipyridyl) [Ru(bpy)₃²⁺], which oxidizes guanine bases in DNA as follows:
Ru(bpy)₃²⁺=Ru(bpy)₃³⁺+e⁻ (1)
Ru(bpy)₃³⁺+DNA(guanine)→Ru(bpy)₃²⁺+DNA(guanine⁺) (2)

DNA(guanine⁺) may be further oxidized. Cycling of Ru(bpy)₃³⁺ by the fast chemical step in equation 2 may provide a catalytic current in the voltammetry that is enhanced compared to either that of Ru(bpy)₃²⁺ or DNA alone. The peak current may depend upon the rate of this chemical step.

The double-helix structure of DNA shields guanine from contact with Ru(bpy)₃³⁺, or from other catalytic oxidants. When the double helix is disrupted, for example, by DNA damage and/or DNA adduct formation, the guanine bases become more available and react more rapidly with the Ru(bpy)₃³⁺. This exposure of guanine leads to increases in the catalytic current. In this manner, the intact double-stranded DNA may be distinguished from damaged double-stranded DNA.

In addition to Ru or Co, the metal center of the metal complex may be osmium, rhenium, iron, or a combination comprising one or more of the foregoing metal centers. In addition to bpy, other suitable ligands include are well known in the art and include, but are not limited to, NH₂; NHR; NRR′; pyridine; pyrazine; isonicotinamide; imidazole; substituted derivatives of bipyridine; terpyridine (“tpy”), 2,3-bis(2-pyridyl)pyrazine (“dpp”); 2,3,5,6-tetrakis(2-pyridyl)pyrazine; and 2,2′-bipyridimidine (“bpm”) and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam); isocyanide; and 2,3-bis(2-pyridyl)quinoxaline. R and R′ are independently hydroxy, alkyl alkoxy, and acyl. Substituted derivatives, including fused derivatives, may also be used. In some cases, porphyrins and substituted derivatives of the porphyrin family may be used. Typically, compounds having a relatively high oxidation potential, e.g. >0.7 V, are effective to oxidize guanine.

In the catalytic oxidation method, the oxidation probe may be a redox polymer which may be present in the double-stranded DNA layer or present as an additional layer as described previously.

In the catalytic oxidation method, after contacting with the oxidation probe, the biosensor is scanned to positive voltages and increases in the SWV peaks indicate that the DNA in the films has been damaged.

In another method, the metal complex may be an electroactive probe which binds the DNA. The electroactive probe binds more strongly to intact double-stranded DNA than to damaged DNA. Such an electroactive probe is tris-(2,2′-bipyridyl)cobalt (III) [Co(bpy)³⁺]. Other electroactive probes may be similar to the previously described oxidation probes so long as they preferentially bind to intact double-stranded DNA over damaged DNA. After contacting with the electroactive probe, the biosensor may be scanned for positive voltages, wherein decreased SWV peaks indicate that the DNA in the films has been damaged.

Voltammetric readings may be taken after a certain time period (e.g., about 5 to about 15 minutes), or may be taken at intervals to monitor the time course of the reaction.

As an alternative to voltammetry, other methods may be used to detect DNA damage. Suitable methods include, for example, HPLC-MS-MS, capillary electrophoresis and electrochemiluminescence (ECL).

In the ECL method, a metal complex or redox polymer attached to DNA can provide a sensitive means of detection. In an example of the ECL process, Ru(bpy)₃²⁺ is photoexcited to [Ru(bpy)₃²⁺]*. The decay of [Ru(bpy)₃²⁺]* to the ground state at 610 nm is measured in the detection step. The method of ECL detection thus comprises generating a photoexcited metal complex wherein the metal complex is covalently attached to double-stranded DNA in the double-stranded DNA layer, and detecting decay of the photoexcited metal complex to the ground state. Detecting may comprise detecting luminescent emission. The metal complex and/or redox polymer can be one of those described previously. An advantage of ECL detection is that ECL can be achieved by direct reaction of a metal complex or redox polymer with DNA. While an optional reductant such as triproylamine may be employed, the reductant does not appear to be required. Without being held to theory, the ECL response is believed to involve guanines in the DNA. The ECL response is greater in ss-DNA than ds-DNA, making it sensitive to the hybridization state of the DNA. Also, the ECL response is greater for damaged DNA than for intact double-stranded DNA.

A biosensor or plurality of biosensors may be provided in the form of an array. The array may be present, for example, on a substrate or solid support. By “substrate” or “solid support” is meant a material that can be modified to contain discrete individual sites (including wells) appropriate to the formation or attachment of electrodes. The substrate may be a single material (e.g., for two dimensional arrays) or may be layers of materials (e.g., for three dimensional arrays). Suitable substrates include metal surfaces such as glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon®, and derivatives thereof, etc.), GETEK (a blend of polypropylene oxide and fiberglass), etc, polysaccharides, nylon or nitrocellulose, resins, silica or silica-based materials including silicon and modified silicon, carbon, metals, inorganic glasses, a variety of other polymers, and combinations comprising one or more of the foregoing materials.

A kit for toxicity screening includes one or more biosensors as described herein. A plurality of biosensors may be provided in the form of an array. The biosensor or array of biosensors may be provided on a solid support. The kit may include appropriate buffers, detection reagents and other solutions and standards for use in the assay methods described herein. In addition, the kits may include instructional materials containing directions (i.e., protocols) for the practice of the toxicity screening method. While the instructional materials typically comprise written or printed materials, they are not limited to such. A medium capable of storing such instructions and communicating them to an end user may be employed. Such media include, but are not limited to electronic storage media (e.g., magnetic discs, tapes, cartridges, chips), optical media (e.g., CD ROM), and the like. Such media may include addresses to internet sites that provide such instructional materials.

An instrument for performing toxicity screening is also included. The instrument can be designed for simple and rapid incorporation into an integrated assay device, e.g., a device comprising, in communication, an electrochemical detector (e.g. square wave voltammetry, or electrochemiluminescence) circuitry, appropriate plumbing for administration of a sample, and computer control system(s) for control of sample application, and analysis of signal output. The instrument is designed to employ a biosensor as described herein. The instrument may be designed to employ a plurality of biosensors in the form of, for example, an array. The biosensor or array of biosensors may be provided on a solid support. Automated or semi-automated methods in which the biosensors are mounted in a flow cell for addition and removal of reagents, to minimize the volume of reagents needed, and to more carefully control reaction conditions, may be employed.

In another embodiment, the disclosed biosensors can be employed to evaluate which enzymes, particularly liver enzymes, form metabolites that damage DNA. In addition, the relative rates of toxic metabolite production can be measured. In these methods, the protein layer of the biosensors comprises a test enzyme. Instead of a test compound, a compound known to be able to produce metabolites which damage DNA are employed. The known compound may be, for example, styrene. It has been shown that cytochrome P450 and myoglobin catalyze the conversion of styrene to styrene oxide, which causes DNA damage. Double-stranded DNA damage produced by the styrene oxide can be measured by a suitable method to detect DNA damage as discussed above.

The invention is further illustrated by the following non-limiting examples.

### EXAMPLES

### Example 1

### Formation of Films

Chemicals and materials. DNA was calf thymus (CT) ds-DNA (Sigma, type XV, 13,000 avg. base pairs, 41.9% G/C) and salmon testes (ST) ds-DNA (Sigma, about 2,000 avg. base pairs, 41.2% G/C). Horse heart myoglobin (Mb) was from Sigma (MW 17,400) dissolved in pH 5.5 buffer and filtered through an Amicon YM30 membrane (30,000 MW cutoff). Pseudomonas putida cyt P450_{cam} (MW 46,500) was expressed in E. Coli DH5α containing P450_{cam} cDNA and purified. Poly(diallydimethylammonium chloride) (PDDA), toluene and benzaldehyde were from Aldrich. Deoxyribonuclease I (Type IV: from Bovine Pancreas), phosphodiesterase I, alkaline phosphatase, styrene and styrene oxide were from Sigma. Sodium dodecyl sulfate (SDS) was from Acros. Water was treated with a Hydro Nanopure system to specific resistance>16 mΩ-cm. All other chemicals were reagent grade.

Film construction. Films were grown on PG disks (pyrolytic graphite) that had been abraded on 400 grit SiC paper, then with coarse Emery paper (3M Crystal Bay), then ultrasonicated in water for 30 seconds. Layers of ds-DNA and protein were adsorbed alternately for 15 minutes onto the rough PG electrodes, and washed with water between adsorption steps. Adsorbate solutions were: (a) 2 mg mL⁻¹ DNA in 5 mM pH 7.1 TRIS buffer and 0.50 M NaCl; (b) 2 mg mL⁻¹ PDDA in 50 mM NaCl; (c) 3 mg mL⁻¹ Mb in 10 mM pH 5.5 acetate buffer; and (d) 1 mg mL⁻¹ cyt P450_{cam} in the pH 5.5 buffer. Film of architecture denoted PDDA/DNA(/Mb/DNA)₂ or PDDA/DNA(/cyt P450_{cam}/DNA)₂ were employed.

Film assembly was monitored at each step with a quartz crystal microbalance (QCM, USI Japan) using 9 MHz QCM resonators (AT-cut, International Crystal Mfg.). To mimic the carbon electrode surface used for voltammetry, a partly negative monolayer was made by treating gold-coated (0.16±0.01 cm²) resonators with 0.7 mM 3-mercapto-1-propanol and 0.3 mM 3-mercaptopropionic acid in ethanol. Films were assembled as for PG electrodes. Resonators were dried in a stream of nitrogen before measuring the frequency change (ΔF). Absorbed mass was estimated with the Sauerbrey equation. For 9 MHz quartz resonators, the dry film mass per unit area M/A is:
M/A(g cm⁻²)=−ΔF(Hz)/(1.83×10⁸) (3)
The nominal thickness (d) of dry films was estimated with an expression confirmed by high resolution electron microscopy:
d(nm)≈(−0.016±0.002)ΔF(Hz) (4)

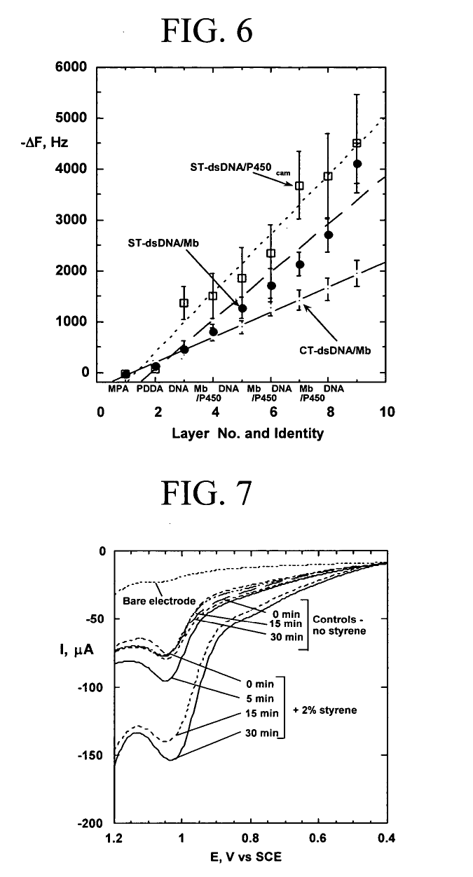

QCM Monitoring of Film Assembly. Films of architecture DNA(/protein/DNA)₂ (see graphical abstract) were used to strike a balance between high enzyme loading favorable for the catalytic conversion and mass transport limitations that occur with thicker films. QCM frequency shifts measured during growth of the films were nearly linear (FIG. 6), suggesting regular film growth with reproducible layers of DNA and proteins. (Avg. values for 9 [Mb/CT ds-DNA,] (◯), 5 [Mb/ST ds-DNA] (●) and 4 [cyt P450_{cam}/ST ds-DNA] (□) replicate films).

ΔF-values and equation 3 were used to obtain weights of protein and DNA. Equation 4 was used to estimate the average nominal thickness of the films (Table 1).

PDDA/DNA(Cyt P450_{cam}/DNA)₂ films were the thickest consistent with the larger size of cyt P450_{cam} compared to Mb. Cyt P450_{cam} layers also adsorbed the most DNA, even though the isoelectric pH of Cyt P450_{cam} is 4.6 and the protein is slightly negative under our adsorption conditions. This behavior is consistent with previously observed binding properties of this enzyme, which utilizes localized surface patches of both negative and positive charge. PDDA/DNA(Mb/ST-ds-DNA)₂ films contained more protein and DNA and were 50% thicker than PDDA/DNA(Mb/CT-ds-DNA)₂ films (Table 1).

TABLE 1
Average characteristics of protein/DNA films from QCM results

| | thickness, | wt. DNA, | wt. protein, |
|---|---|---|---|
| film | nm | μg cm⁻² | μg cm⁻² |
| DNA(Mb/CT-ds-DNA)₂ | 20 | 3.1 | 3.6 |
| DNA(Mb/ST-ds-DNA)₂ | 30 | 5.8 | 4.9 |
| DNA(Cyt P450_{cam}/ST-ds-DNA)₂ | 40 | 15 | 2.9 |

### Example 2

### Generation of Metabolites

Incubation of films with styrene. When myoglobin or cyt P450_{cam} in polyion films are activated by hydrogen peroxide, styrene is converted to styrene oxide. The iron heme in the protein is oxidized by hydrogen peroxide to an oxyferryl intermediate, which transfers an oxygen atom to the olefinic double bond of styrene. This reaction proceeds when DNA is the polyion. Even though styrene oxide reacts with DNA, it can be detected in significant amounts after 1 hour reaction. Hydrogen peroxide was added to solutions containing styrene so that the protein in the film would catalyze production of styrene oxide to react with DNA. Reactions were done at pH 5.5, since this pH was the previously established optimum for reaction of styrene oxide with DNA, and also gives adequate turnover for the epoxidation.

Incubation of DNA/protein films for metabolite generation and reaction with DNA was done in a thermostated vessel at 37° C. containing 1-4% styrene (by volume). To activate the enzymes for styrene oxide formation, 0.2 to 2 mM H₂O₂ was added to 10 mL pH 5.5 buffer containing 50 mM NaCl. After incubation, the electrodes were rinsed with water and transferred to pH 5.5 buffer containing 50 μM Ru(bpy)₃²⁺ for analysis by catalytic SWV. For the probe binding method, washed electrodes were transferred to pH 5.5 buffer containing 20 μM Co(bpy)₃³⁺.

### Example 3

### Voltammetric Response to Metabolite Generation

Voltammetry. CH Instruments 660A and CH 430 electrochemical analyzers were used for square wave voltammetry (SWV), with ˜95% ohmic drop compensated. The thermostated cell employed a saturated calomel reference electrode (SCE), a Pt wire counter electrode, and film-coated working electrode disk (A=0.2 cm²) of ordinary basal plane pyrolytic graphite (PG, Advanced Ceramics). SWV conditions were 4 mV step height, 25 mV pulse height, and 15 Hz frequency. The electrolyte was 10 mM sodium acetate buffer and 20 or 50 mM NaCl, pH 5.5. Solutions were purged with purified nitrogen and a nitrogen atmosphere maintained for voltammetry.

In the first DNA analysis method, square wave voltammetry (SWV) with a small amount of ruthenium tris(2,2′-bipyridyl) [Ru(bpy)₃²⁺], which increases sensitivity by catalytically oxidizing guanines in DNA (eqs 3 and 4), was performed. When ds-DNA in the film is damaged by styrene oxide, guanines released from the protection of the double helix as well as adducts of adenine become easily oxidized and the catalytic current (eqs 1 and 2) increases.

After reaction in 0.2 mM hydrogen peroxide/2% styrene, Mb/DNA electrodes were washed, then placed into a solution of 50 μM Ru(bpy)₃⁺. Catalytic SWV oxidation peaks were observed at about 1 V vs. SCE, and peak height (negative peaks, FIG. 7) increased with reaction time of the film. Control electrodes which were incubated in hydrogen peroxide without styrene showed catalytic oxidation peaks of similar heights to freshly prepared films. This confirms that this low concentration of peroxide has a minimal influence on the intact ds-DNA, which still reacts at a finite rate with Ru(bpy)₃²⁺. The peaks for electrodes that had been activated by hydrogen peroxide with styrene present increased with reaction time, presumably because of disruption of the double helix by the formation of adducts of DNA bases with styrene oxide. The heights of these peaks did not depend on styrene concentration between 1-4%. Concentrations of hydrogen peroxide of 2 and 10 mM gave larger peaks, but smaller increases with time and larger control peaks. A hydrogen peroxide concentration of 0.2 mM gave the largest differences between reacted samples and controls.

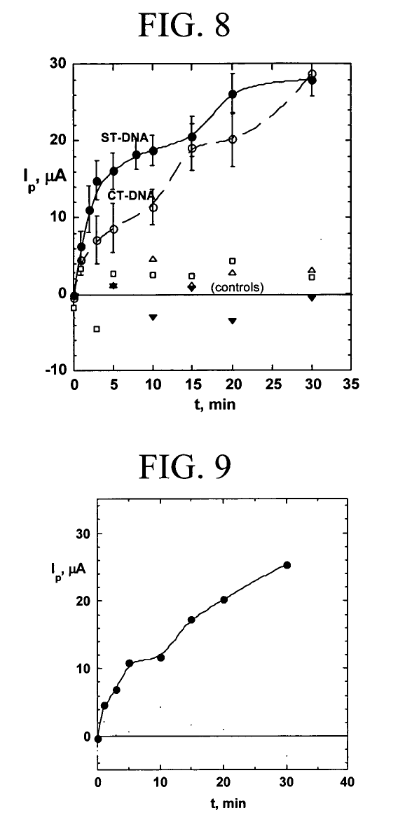

Average SWV catalytic peak currents for ds-DNA/Mb films increased with reaction time at relatively larger rates for the first 5 min, then at lower rates at longer times (FIG. 8). Larger initial rates of peak current increase were found with Mb/ST ds-DNA films than with Mb/CT ds-DNA films. Also shown are controls representing incubation of ST-DNA/Mb films with 2% styrene but no H₂O₂ (□), 2% toluene+0.2 mM H₂O₂ (▾) 0.2 mM benzaldehyde+0.2 mM H₂O₂ (Δ). (Avg. peak current for unreacted DNA/Mb electrodes was subtracted). Error bars in FIG. 8 are mainly the result of film-to-film variability. No significant increases in peak current or trends with incubation time were found when films were incubated with toluene and hydrogen peroxide, styrene alone, benzaldehyde, or hydrogen peroxide alone (FIG. 8, controls).

FIG. 9 shows that similar results were obtained when DNA(Cyt P450_{cam}/ST-ds-DNA)₂ films were incubated with styrene and hydrogen peroxide. Again, a rapid increase in SWV peak current for the first five minutes was followed by a slower increase from 5-30 minutes. However, the rate of increase was about 30% less than with Mb/ST-DNA films (e.g., FIG. 8). Controls with hydrogen peroxide alone (◯) gave no significant increases in peak current.

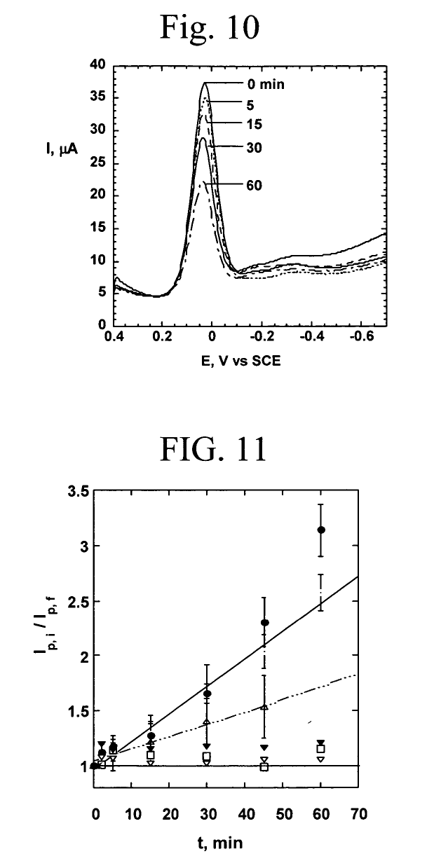

The second DNA-damage detection method employed tris(2,2′-bipyridyl)cobalt (III) [Co(bpy)₃³⁺] as an electroactive probe, which binds more strongly to intact ds-DNA in films compared to DNA damaged by styrene oxide. In these studies, the SWV peak decreased after reaction with 0.2 mM hydrogen peroxide, and 2% styrene were not large enough to be useful. The optimum concentrations were 2 mM hydrogen peroxide and 4% styrene. After incubation of films in this medium for a given time, electrodes were washed and placed into 20 μM Co(bpy)₃³⁺. FIG. 10 shows that the largest peaks for the reduction of Co(bpy)₃³⁺ at 0.04 V vs. SCE were found when the ds-DNA in the film was intact, because the probe binds in largest amounts to these films. (SWV Amplitude: 25 mV; Frequency: 15 Hz; Step: 4 mV; One electrode was used for each assay). The peak height decreased with incubation time, suggesting the gradual loss of the film's ability to bind the probe. The small peak at about −0.3 V vs. SCE represents the Fe^{III}/Fe^{II} reduction of the protein.

The time course of the reaction can readily be monitored via the ratio of initial SWV peak current for a given film (I_{p,i}) to that after reaction (I_{p,f}). With Mb in the films, similar linear increases of the I_{p,i}/I_{p,f} ratio with time were observed over 45 min. (FIG. 11). Also shown are controls representing incubation of ST-DNA/Mb films with 4% styrene but no hydrogen peroxide (□) 4% toluene+2 mM hydrogen peroxide (▾) 50 μM benzaldehyde with no hydrogen peroxide (∇). Error bars represent standard deviations for 5 replicates. In this case, ST- and CT-ds-DNA showed similar slopes of peak ratio vs. reaction time (solid line) with Mb in the film. Films containing cyt P450_{cam} gave smaller slopes of peak ratio vs. time (dashed line), perhaps because of smaller molar amounts of active enzyme in these films compared to Mb films. Control experiments involving incubation of films in toluene and hydrogen peroxide, benzaldehyde, or hydrogen peroxide alone gave no significant increases or trends in peak current ratios with incubation time. (FIG. 11).

### Example 4

### Confirmation of DNA-Styrene Oxide Adducts by Capillary Electrophoretic Analysis

Capillary electrophoretic analysis. A Beckman PACE 5000 with UV detector at 254 nm was used for capillary electrophoresis (CE) to analyze hydrolyzed DNA that had been reacted with styrene oxide. 1 mg mL⁻¹ ST ds-DNA in 50 mL buffer or (PDDA/DNA)₂ films on 3×10 cm carbon cloth in solution were reacted in 50 mL buffer and 0.87 mmol styrene oxide at 37° C. with stirring. Reacted DNA in solution or in films was hydrolyzed by incubating with deoxyribonuclease I (0.1 mg per mg of DNA) for 20 hr at 37° C., followed by incubation with phosphodiesterase I (0.01 unit/mg DNA) and phosphatase, alkaline (0.6 unit/mg DNA) for 5 hours at 37° C. The resulting samples, consisting mainly of individual nucleosides, were analyzed using CE with 20 mM pH 7 phosphate buffer and 100 mM SDS. The capillary was 75 μm×50 cm, with a polyimide coating. To make standard styrene oxide adducts, 0.5 mg mL⁻¹ guanosine or adenosine were reacted with styrene oxide.

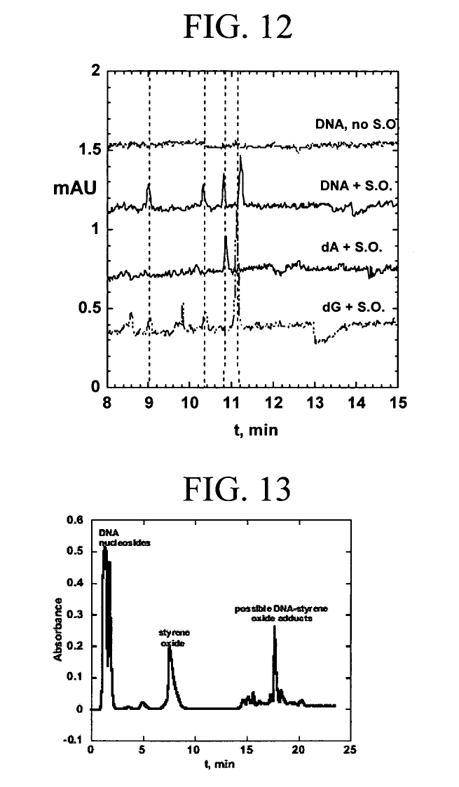

It was previously shown that that styrene oxide is formed from styrene using Mb and cyt P450_{cam} films. However, available capillary electrophoresis and HPLC-MS/MS methods were not sensitive enough to detect and identify damaged DNA directly from the protein-DNA films. Thus, capillary electrophoresis and HPLC-MS were used to confirm that reaction of styrene oxide with DNA in (PDDA/DNA)₂ films and in solution gave the reported DNA-styrene oxide adducts. After incubation of ds-DNA with saturated styrene oxide, DNA was hydrolyzed enzymatically to the individual nucleosides. FIG. 12 demonstrates capillary electrophoretic analyses of ST ds-DNA in a (PDDA/DNA)₂ on carbon cloth reacted with styrene oxide. Compared with the capillary electropherograms of undamaged hydrolyzed ST ds-DNA that showed only the 4 unreacted DNA nucleosides, there were new peaks evident at retention times (t_{R}) between 8 and 12 min. Dashed lines identify peaks common to reacted samples of DNA and dG or dA. The much larger peaks of unreacted nucleosides occur at t_{R}<8 min.

Peaks for the damaged hydrolyzed DNA at t_{R} 9.0, 10.4, and 11.2 min also appeared in a deoxyguanosine sample that had been reacted with styrene oxide. The peak at t_{R} 10.8 min corresponds to a peak in a deoxyadenosine sample that had been reacted with styrene oxide. Similar results were obtain when DNA was in solution or in these films.

### Example 5

### Confirmation of DNA-Styrene Oxide Adducts by HPLC-MS/MS

Liquid chromatography (LC)-mass spectrometry (MS). A Perkin-Elmer LC with diode-array (255 nm and 280 nm) and mass spectrometer (Micromass, Quattro II) detection were used. Full-scan spectra were taken at low cone voltage (15 V) in the positive ion mode (ESI). The HPLC column was Restek Ultra C-18 reversed-phase (i. d. 2.1 mm, length 10 cm, particle size 5 μm). Solvents were A: 5% acetonitrile/95% water with 0.05% TFA and B: 100% acetonitrile with 0.05% TFA. The gradient at 300 μL/min was 100% A for 5 min, ramped to 100% B in 10 min, then 100% B for 10 min. Multiple reaction monitoring (MRM) MS/MS mode employed Argon at 1.7×10⁻³ mm Hg, cone voltage 15 V, and collision energy 15 eV.

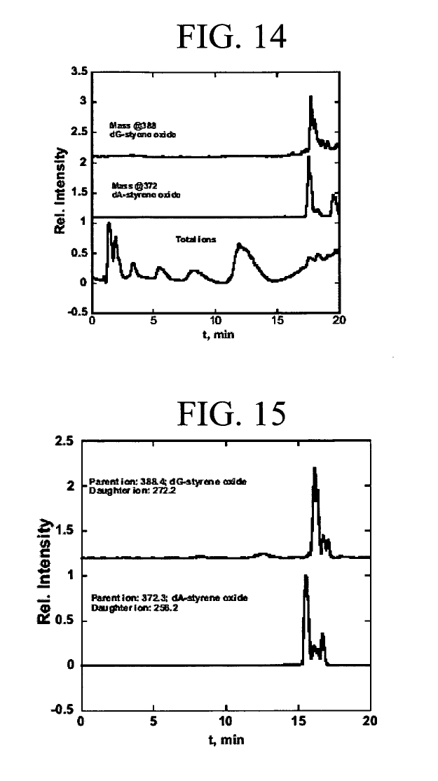

Confirmatory results were obtained by HPLC-MS/MS. UV-HPLC of damaged DNA showed the suspected dG and dA adducts as a series of peaks at t_{R} 15-21 min, with the major peak in this group at 18 min (FIG. 13). Electrospray ionization mass spectral detection with display of mass at 388 M/e, corresponding to dG-styrene oxide adducts (FIG. 14), showed a major peak at t_{R} 18 min, and the 372 M/e display, corresponding to dA-styrene oxide adducts, gave a major peak at about 17.8 min. In multiple reaction monitoring (MRM) mode, the most significant daughter ions for both of these peaks corresponded to the loss of a sugar group (M/e=116) from the parent ions (FIG. 15). These experiments showed at least 4 styrene oxide adducts each for dA and dG. These results confirm the formation of dA-styrene oxide and dG-styrene oxide adducts under reaction conditions similar to those used for the films. However, the sensitivity limitations of these methods required reaction times much longer than for the SWV methods.

### Example 6

### Biosensor Using [Ru(bpy)₂poly(4-vinylpyridine)₁₀Cl]⁺ as a Catalyst

Film Assembly. Basal plane PG electrodes were polished with 400-grit SiC paper and then ultrasonicated for 30 seconds each in ethanol and then in water. Electrodes were dried in nitrogen and dipped into 6 mg mL⁻¹ PSS containing 0.5 M NaCl for 15 min. After washing with water, these PG electrodes were then dipped into 0.05% (m/V) [Ru(bpy)₂(PVP)₁₀Cl)]Cl (denoted hereafter as CIRu-PVP) in 5% ethanol/water for 15 minutes. Adsorption for 15 min provides steady-state adsorption of PSS on oppositely charged surfaces.^{18, 19}

After fabricating PSS/CIRu-PVP films, electrodes were dipped into 2 mg mL⁻¹ CT ds-DNA in pH 7.0 Tris buffer containing 0.5 M NaCl for 15 min, rinsed with water, and then dipped into 2 mg mL⁻¹ PDDA. Alternate adsorption cycles were repeated until the desired number of layers were made. Final film structure is denoted as PSS/CIRu-PVP/CT-ds-DNA/PDDA/CT-ds-DNA.

For films containing Mb, PG electrodes were abraded with 400-grit SiC paper, rinsed with water, further roughened using medium Crystal Bay emery paper (PH4 3M 001K), and then ultrasonicted in ethanol and water successively for 30 seconds. Roughening of the PG surface provides films with more active Mb. Additional steps were the same as above except PDDA was replaced with 3 mg mL⁻¹ Mb in pH 5.5 buffer. Final film structure is denoted as PSS/CIRu-PVP/CT-ds-DNA/(Mb/CT-ds-DNA)₂. Electrochemical surface area was estimated at 0.21 cm² using peak currents of soluble ferricyanide and the Randles-Sevcik equation.

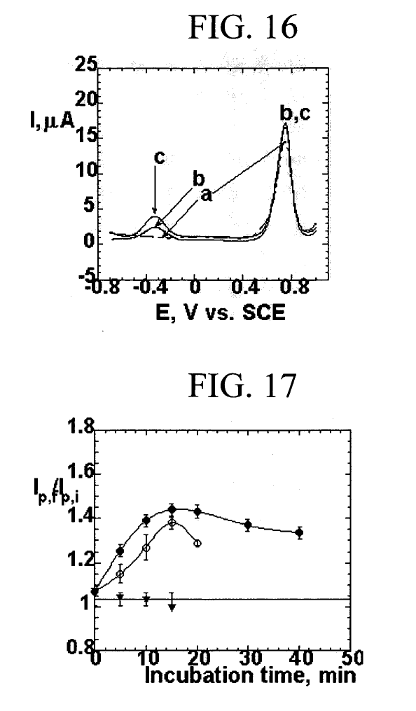

FIG. 16 shows SWVs of Ru/Mb/DNA films on rough PG. Roughening the PG increased the amount of Mb and CIRu-PVP in the film by increasing the electrode surface area. When Mb/DNA layers were grown on top of PSS/CIRu-PVP, an obvious increase in the peak current at 0.75 V was found (FIG. 16, curve b), suggesting catalytic oxidation of the DNA. Also, a peak corresponding to the MbFe^{III}/MbFe^{II} redox couple appeared at −0.3 V. In the presence of oxygen, an increase in the Mb peak due to the catalytic reduction of oxygen was observed, while there is no change in the Ru^{III}/^{II} peak (FIG. 16, curve c). This suggests that Ru^{III}/^{II} and MbFe^{III}/^{II} redox peaks are independent of one another, and Mb does not influence the catalytic peak current for CIRu-PVP.

H₂O₂ activates Mb to oxidize styrene to styrene oxide. After incubating Ru/Mb/DNA films with saturated styrene and H₂O₂ for different times, the SWV peaks at 0.75 V increased. The ratio of final peak current after incubation to initial peak current of CIRu-PVP (FIG. 17) showed an increase over the first 15 minutes and then decreased gradually. An H₂O₂ concentration of 2 mM gave slightly larger peaks than 0.2 mM. Films incubated in toluene and H₂O₂ did not show increases in the peak currents (FIG. 17).

To verify the formation of styrene oxide, a Ru/Mb/DNA film was incubated for 75 minutes with saturated styrene and 2 mM H₂O₂. The buffer was then extracted with hexane and the extract analyzed by GC. A peak at retention time 6.6 min was found, which indicated with standardization that 58 nmol of styrene oxide was formed.

### Example 7

### ECL Detection of DNA Damage

Film assembly. DNA-redox polymer films were constructed by the layer-by-layer electrostatic assembly method. Basal plane PG electrodes were polished with 400 grit SiC paper and then with 0.3 μm α-alumina slurries on Buehler Microcloth, washed with water, sonicated in ethanol for 15 minutes, and then sonicated in water for 15 minutes. Layers were constructed by placing a 30 μL drop of 0.2% aqueous [Ru(bpy)₂(PVP)₁₀](CIO₄)₂ onto each PG electrode, allowing 15 minutes to achieve saturated adsorption and then washing with water. Subsequently, 30 μL of DNA solution (2 mg mL⁻¹ DNA in 5 mM pH 5.5 acetate buffer+0.05 M NaCl) was place on this PG surface, allowed to adsorb 15 min, and then washed with water. This sequence was repeated to obtain films with 2 redox polymer/DNA bilayers. Films containing ss-DNA and other polynucleotides were also assembled in this way. Assembly of films was monitored at each step with a quartz crystal microbalance.

Reactions with Styrene Oxide. Incubations of films were done in styrene oxide solutions in a stirred reactor at 37.0±0.5° C. A 120 μL volume of neat styrene oxide or toluene (as control) was added to 10 mL of acetate, pH 5.5, +50 mM NaCl to give saturated solutions. pH 5.5 gave optimum reaction rates of DNA with styrene oxide and also allowed efficient ECL production. PG electrodes coated with polynucleotide or DNA films were incubated in the stirred emulsion and then rinsed with water and transferred to the electro-chemical cell containing pH 5.5 buffer for SWV/ECL analysis.

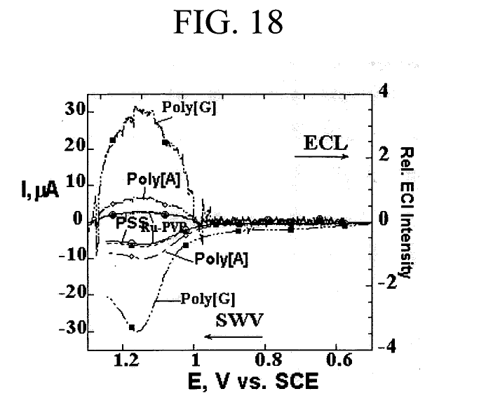

FIG. 18 shows that combined ECL/SWV measurements on films containing Ru-PVP, alone or in (PVP/PSS)₂ films, gave the Ru^{II}/Ru^{III} oxidation peak and a very small amount of light. However, (Ru-PVP/poly[G])₂ films gave a significant ECL peak, as well as a catalytic current by SWV that was much larger that a noncatalytic RU^{II}/Ru^{III} oxidation peak for Ru-PVP films not containing poly[G]. FIG. 18 also shows that (Ru-PVP/poly[A])₂ gave a small catalytic current and a very small ECL signal, slightly above the background for Ru-PVP films.

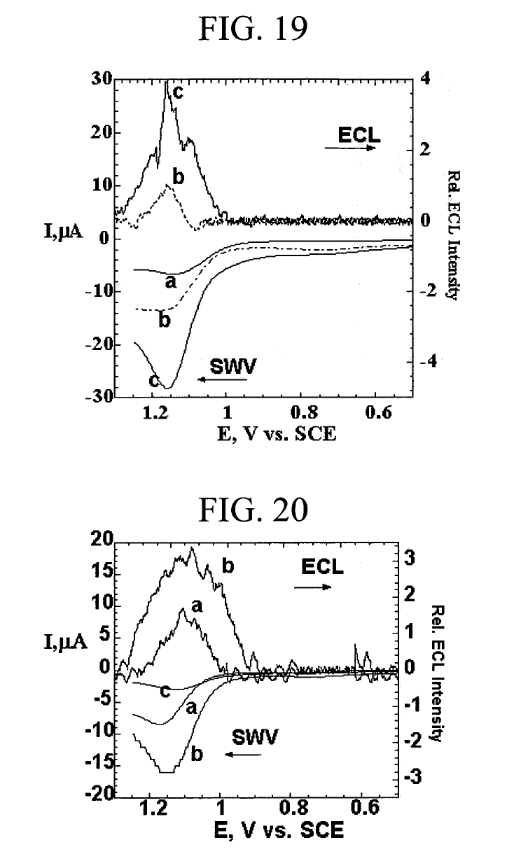

The influence of hybridization on the ECL signal was investigated by using films containing hybridized and unhybridized poly[G]. FIG. 19 compares the ECL/SWV responses of films of (Ru-PVP/poly[G])₂ and (Ru-PVP/poly[G]/poly[C])₂. The latter films were made by using a solution of poly[G] and poly[C] for which UV-Vis spectra confirmed hybridization. Both ECL and SWV peaks are about 3-fold larger for films containing the hybridized poly[G]/poly[C] layers compared to the film with only the poly[G] layer (FIG. 14).

Similar results were obtained when comparing ECL/SWV signals for films containing ss- and ds-DNA (FIG. 20). Films containing ss-DNA gave about twice the ECL signal as those made with ds-DNA. SWV peaks for the ss-DNA films were about 2.5-fold higher than their ds-DNA analogues. Similar results were obtained for calf thymus and salmon testes DNA. Films assembled with DNA and the polycation PDDA showed no significant ECL peaks.

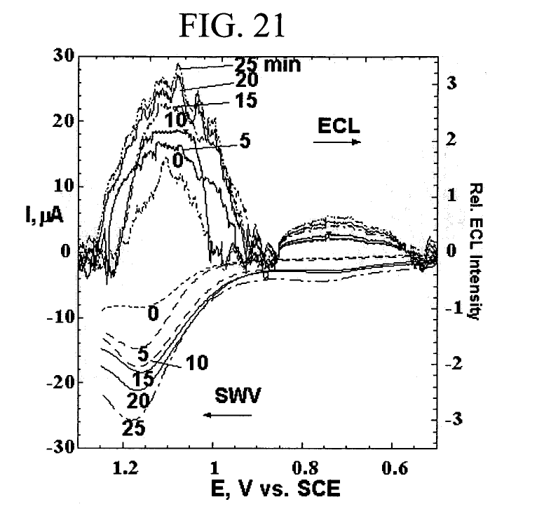

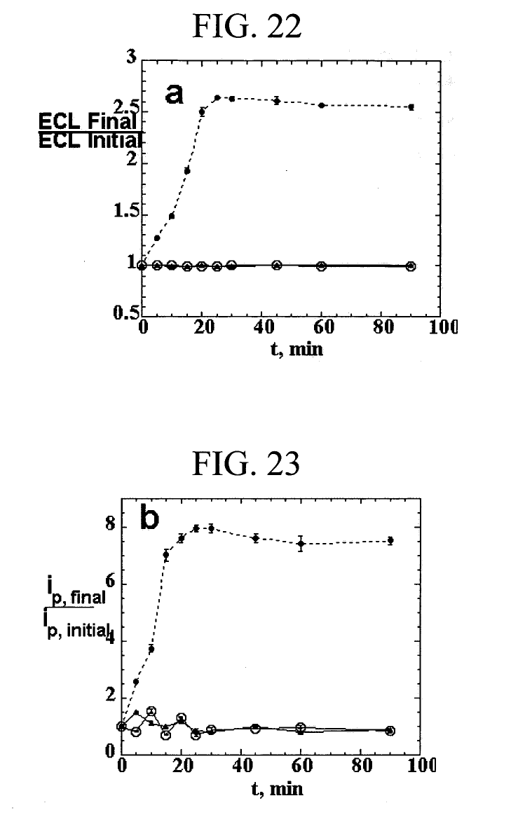

When (Ru-PVP/ds-DNA)₂ films were incubated with styrene oxide and then scanned by SWV, increases in the ECL and the SWV peaks were observed with increasing incubation time (FIG. 21). Average peak currents for the ds-DNA films increased linearly with incubation time for about the first 20 minutes, followed by a slight decrease (FIG. 22, 23). When (Ru-PVP/ds-DNA)₂ films were incubated with toluene, for which no chemical reactions with DNA have been reported, or in buffer only, ECL and SWV peaks remained within electrode-to-electrode variability and showed no trends with incubation time. Error bars in FIGS. 22, 23 are mainly the result of electrode-to-electrode variability. However, both the error bars and scatter in the controls were smaller for the ECL rations than for the SWV ratios.

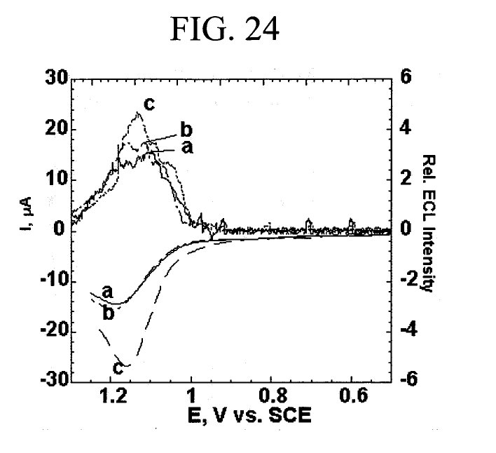

In addition to catalytic oxidation of guanines, it is possible that adducts formed on DNA by reaction with styrene oxide could be catalytically oxidized by the ruthenium metallopolymer. To assess this possibility, styrene oxide was incubated with films containing individual polynucleotides and the metallopolymer. FIG. 24 shows that both ECL and SWV peaks increased after 10 minutes of incubation of (Ru-PVP/poly[G])₂ with styrene oxide. An 80% increase in SWV peak current and a 40% increase in ECL intensity was found. However, for films incubated with toluene, ECL and SWV peaks were nearly identical to initial values.

The novel biosensor described herein comprises a double-stranded DNA layer a protein layer, wherein the protein layer comprises an enzyme that converts compounds into metabolites that damage double-stranded DNA. The biosensor may be in the form of a single electrode or an array of electrodes. The biosensor may be advantageously employed in methods of determining toxicity of a test compound. The disclosed methods are readily automated and are faster and more reproducible than conventional microbiological testing.

All ranges disclosed herein are inclusive and combinable. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

1. A biosensor, comprising:
an electrode;
a first polynucleotide layer comprising a double-stranded polynucleotide, wherein the electrode and the polynulceotide layer are in electrochemical communication; and
a first protein layer in chemical communication with the polynucleotide layer, wherein the protein layer comprises a protein that converts a genotoxic compound into a metabolite that damages double-stranded DNA.

2. The biosensor of claim 1, wherein the double-stranded polynucleotide comprises double-stranded DNA.

3. The biosensor of claim 1, wherein the first polynucleotide layer has a first side and a second side, wherein the first side of the first polynucleotide layer is disposed on and in intimate contact with the electrode; and
wherein the first protein layer has a first side and a second side, wherein the first side of the first protein layer is disposed on and in intimate contact with the second side of the first polynucleotide layer.

4. The biosensor of claim 1, wherein the first protein layer comprises a cytochrome P450, myoglobin, hemoglobin, a peroxidase, or a combination comprising one or more of the foregoing enzymes.

5. The biosensor of claim 1, further comprising a polyion layer disposed between the electrode and a first side of the first polynucleotide layer, wherein the polyion is not a polynucleotide.

6. The biosensor of claim 5, wherein the polyion is poly(diallyldimethylammonium chloride, poly(ethylene)imine, or a combination comprising one or more of the foregoing polyions.

7. The biosensor of claim 1, further comprising a redox polymer layer disposed between the electrode layer and a first side of the first plynucleotide layer.

8. The biosensor of claim 7, wherein the redox polymer layer comprises [Ru(bpy)₂(PVP)₁₀](ClO₄)₂, [Ru(bpy)₂poly(4-vinylpyridine)₁₀Cl]⁺, or a combination comprising one or more of the foregoing redox polymers.

9. The biosensor of claim 1, further comprising:
a second polynucleotide layer comprising the double-stranded polynucleotide, the second polynucleotide layer having a first side and a second side, wherein the first side of the second polynucleotide layer is disposed on and in chemical communication with a second side of the first protein layer; and
a second protein layer having a first side and a second side, wherein the first side of the second protein layer is disposed on and in chemical communication with the second side of the second polynucleotide layer, wherein the second protein layer comprises a second protein that converts the genotoxic compound into a metabolite that damages double-stranded polynucleotides, wherein the second protein is the same or different as the first protein.

10. An array comprising a first biosensor as in claim 1 and a second biosensor as in claim 1, wherein the first and second biosensors are disposed on a solid support, and wherein the first biosensor and the second biosensor are the same or different.

11. A method of determining the genotoxicity of a test compound, comprising:
contacting the biosensor of claim 1 with the test compound; and
detecting double-stranded polynucleotide damage in the polynucleotide layer resulting from said contacting.

12. The method of claim 11, wherein the first protein layer comprises a cytochrome P450, myoglobin, hemoglobin, a peroxidase, or a combination comprising one or more of the foregoing enzymes.

13. The method of claim 11, wherein the biosensor further comprises a polyion layer disposed between the electrode and a first side of the first polynucleotide layer, wherein the polyion is not a polynucleotide.

14. The method of claim 13, wherein the polyion is poly(diallyldimethylammonium chloride, poly(ethylene)imine, or a combination comprising one or more of the foregoing polyions.

15. The method of claim 11, wherein the biosensor further comprises a redox polymer layer disposed between the electrode layer and a first side of the firs polynucleotide layer.

16. The method of claim 15, wherein the redox polymer layer comprises [Ru(bpy)₂(PVP)₁₀](ClO₄)₂, [Ru(bpy)₂poly(4-vinylpyridine)₁₀Cl]⁺, or a combination comprising one or more of the foregoing redox polymers.

17. The method of claim 11, wherein detecting is by square wave voltammetry.

18. The method of claim 11, further comprising contacting the biosensor with a metal complex prior to detecting.

19. The method of claim 18, wherein the metal complex is an oxidation probe.

20. The method of claim 11, further comprising contacting the biosensor with hydrogen peroxide prior to detecting.

21. The method of claim 11, wherein detecting is by electrochemiluminescence.
